(19) United States
(12) Patent Application Publication
(10) Pub. No.: US 2018/0066968 A1
(43) Pub. Date: Mar. 8, 2018

(54) FLUIDIC OSCILLATION FLOWMETER WITH SYMMETRICAL MEASUREMENT ORIFICES FOR A DEVICE FOR MONITORING OXYGEN THERAPY

(71) Applicant: L'Air Liquide, Société Anonyme pour I'Etude et I'Exploitation des Procédés Georges Claude, Paris (FR)

(72) Inventor: Fouad AMMOURI, Massy (FR)

(21) Appl. No.: 15/693,609

(22) Filed: Sep. 1, 2017

(30) Foreign Application Priority Data
Sep. 2, 2016 (FR) 1658167

(51) Int. Cl. G01F 1/32; A61M 16/00; A61M 16/10

(52) U.S. Cl. CPC G01F 1/3227; A61M 16/0006; A61M 2205/3334; A61M 2016/003; A61M 2202/0208; A61M 16/1005

(57) ABSTRACT

The invention relates to a fluidic oscillation flowmeter comprising a stabilization chamber (1) comprising a flow-stabilizing element (11), an oscillation chamber (2) comprising a reflux element (21) configured to create at least one oscillating gaseous vortex in the oscillation chamber (2), said reflux element (21) being arranged between two parallel walls (28, 29) delimiting the oscillation chamber (2), a connection conduit (3) fluidically connecting the stabilization chamber (1) to the oscillation chamber (2), and a plane of symmetry (P) separating the connection conduit (3), the stabilization chamber (1), the flow-stabilizing element (11), the fluidic oscillation chamber (2) and the reflux element (21) into two equal and symmetrical parts with respect to said plane of symmetry (P). One of the two parallel walls (28, 29) comprises two measurement orifices (24, 25) arranged symmetrically with respect to the plane of symmetry (P), and the connection conduit (3) has a rectangular cross section. Device for monitoring oxygen therapy, comprising such a fluidic oscillation flowmeter, and oxygen therapy equipment comprising a source of respiratory gas, a gas distribution interface and such a monitoring device.

### CROSS REFERENCE TO RELATED APPLICATIONS

This application claims the benefit of priority under 35 U.S.C. § 119 (a) and (b) to French Patent Application No. 1658167 filed Sep. 2, 2016, the entire contents of which are incorporated herein by reference.

### BACKGROUND

The present invention relates to a fluidic oscillation flowmeter usable in oxygen therapy, to a device intended for monitoring oxygen therapy and equipped with such a fluidic oscillation flowmeter, and to associated oxygen therapy equipment.

In the context of oxygen therapy provided to a patient at home, a monitoring device is normally used which is inserted between the gas source, typically a source of oxygen, and the patient, in such a way as to permit monitoring of the oxygen consumed by the patient and to ensure that the latter is indeed observing his or her treatment. Such a device may be equipped with a communication module for remote transmission of data, for example to a remote server.

Thus, WO-A-2009/136101 describes a device for monitoring oxygen therapy of a patient being treated at home by administration of oxygen, said device comprising a casing with a conduit passing through it, one or more pressure sensors, a microprocessor, a memory, a battery for providing electric current, and a radiofrequency antenna.

EP-A-2670463 proposes a similar device additionally including an accelerometer for monitoring the variable oxygen requirements of the patient depending on the patient's physical activity, in particular normal or sustained activity, low activity or rest, or sleep for example.

Moreover, EP-A-2506766 discloses a device for monitoring the respiration of a patient, comprising a differential pressure sensor arranged on a gas conduit additionally comprising an internal configuration of the Venturi type. This device is principally intended for the detection of apnoea or hypopnea in a patient treated by continuous positive airway pressure.

In addition, EP-A-2017586 proposes a device for monitoring the respiration of a patient under normal ventilation or continuous positive airway pressure. It comprises a gas conduit equipped with a diameter-reducing element generating a pressure drop, and a differential pressure sensor for determining the pressure and the flow rate of the gas.

However, these known devices are not ideal since they are in some cases bulky, consume a lot of energy, are sensitive to drift of the sensors, are imprecise in estimating the volume of gas supplied to the patient, especially in the case of low flow rates, or are sensitive to the pressure variations of the source, etc.

In view of this, the problem addressed is to be able to improve the determination of the flow rate of the gas administered to the patient and which preferably has low electricity consumption and is compact, that is to say miniaturized, and inexpensive.

### SUMMARY

The solution of the invention is therefore a fluidic oscillation flowmeter which is usable in oxygen therapy and which in particular is intended to form part of a device for monitoring a patient, comprising:
a stabilization chamber comprising a flow-stabilizing element,
an oscillation chamber comprising a reflux element configured to create at least one oscillating gaseous vortex in the oscillation chamber, said reflux element being arranged between two parallel walls delimiting the oscillation chamber,
a connection conduit fluidically connecting the stabilization chamber to the oscillation chamber, and
a plane of symmetry P separating the connection conduit, the stabilization chamber, the flow-stabilizing element, the fluidic oscillation chamber and the reflux element into two equal and symmetrical parts with respect to said plane of symmetry P,

characterized in that:
one of said two parallel walls delimiting the oscillation chamber comprises two measurement orifices which are arranged symmetrically with respect to the plane of symmetry P and on an axis perpendicular to the plane of symmetry P and are separated from each other by a distance d of between 0.5 mm and 15 mm, and
the connection conduit has a rectangular cross section of width l₀ and height h₀ such that: 6.5·l₀ h₀≧3·l₀, that is to say the height h₀ is greater than or equal to 3 time the width l₀ of the connection conduit but less than or equal to 6.5 times the width l₀ of the connection conduit.

Choosing an upper limit of the depth h₀ of the connection conduit at 6.5 times the width l₀ is associated, firstly, with the existence of a minimum speed limitation in the connection conduit, starting from which the pressure oscillations begin to take effect in the oscillation chamber. Secondly, for a given flow rate, the greater the height h₀ of the connection conduit, the lower the speed inside. Consequently, the amplitude of variation of the pressure at the sensors (which is proportional to the speed in the connection conduit squared) becomes ever smaller and the measurement ever more sensitive to noise. Under these conditions, the quality of the measurement will therefore tend to deteriorate. It is therefore preferable to limit the height h₀ to the abovementioned values in order to avoid or minimize this phenomenon.

Depending on the circumstances, the invention can comprises one or more of the following technical features:
preferably, the connection conduit has a rectangular cross section of width l₀ and height h₀, such that: 6·l₀≧h₀.
preferably, the connection conduit has a rectangular cross section of width l₀ and height h₀, such that: 5.5·l₀≧h₀.
preferably, the connection conduit has a rectangular cross section of width l₀ and height h₀, such that: h₀≧3.1·l₀.
preferably, the connection conduit has a rectangular cross section of width l₀ and height h₀, such that: h₀≧3.5·l₀.
the connection conduit of rectangular cross section has a length L₀ such that 2·l₀≦L₀≦15·l₀.
the two measurement orifices are separated by a distance d of between 0.5 mm and 10 mm, preferably of between 1 mm and 6 mm.
the gas is air, oxygen or an air/oxygen mixture.
the oscillation chamber comprises a peripheral wall connecting the two parallel walls, that is to say the two walls arranged opposite or facing each other, one of which carries the two measurement orifices.
the two parallel walls delimiting the oscillation chamber form the ceiling and the floor of the oscillation chamber, that is to say the two measurement orifices are arranged in the floor or the ceiling.
the flow-stabilizing element is configured to render the speed profile of the gas at the outlet of this element two-dimensional (2D), hence invariable in the direction perpendicular to the plane of the flowmeter, and moreover symmetrical with respect to the plane of symmetry. In fact, the speed profile of the gas arriving at the inlet of this element is often three-dimensional (3D) and asymmetrical. Suddenly changing the direction of flow at the inlet of this element in a rectangular cross section which moreover narrows increasingly towards the connection conduit, which is itself of rectangular cross section, makes it possible to transform the 3D flow into 2D flow. Moreover, the symmetrical geometry with respect to the plane of symmetry of this element also allows the speed profile to be made symmetrical.
the connection conduit conveys the gas from the stabilization chamber to the oscillation chamber by accelerating the speed of the gas, since the rectangular cross section for passage of the gas is smaller than that of the passage arranged in the stabilizing element. Indeed, a gas speed above a minimum value is needed at the inlet of the oscillation chamber in order to trigger the oscillations since, in the absence of a minimum speed, it is not possible to measure the flow rate of gas.
the two measurement orifices are closed, that is to say covered, by a fluidically leaktight membrane. This membrane transmits the pressure variations from the oscillation chamber to the place where the sensors are located, that is to say microphones or pressure sensors.
the reflux element comprises a portion of semi-cylindrical cross section arranged facing the connection conduit.
the stabilization chamber comprises a first inlet orifice and a first outlet orifice, which are arranged on the plane of symmetry P. The gas enters the stabilization chamber through the first inlet orifice and leaves the stabilization chamber through the first outlet orifice.
the oscillation chamber comprises a second inlet orifice and a second outlet orifice, which are arranged on the plane of symmetry (P). The gas enters the oscillation chamber through the second inlet orifice and leaves the oscillation chamber through the second outlet orifice.
the connection conduit fluidically connects the first outlet orifice of the stabilization chamber to the second inlet orifice of the oscillation chamber.
it additionally comprises one or more pressure sensors or microphones attached to said two measurement orifices in such a way as to be able to measure the pressure in the oscillation chamber, preferably microphones.
each measurement orifice is connected to a pressure sensor or to a microphone.
an inlet channel is fluidically connected to the first inlet orifice of the stabilization chamber. The inlet channel supplies the stabilization chamber with gas.
it additionally comprises a casing within which the connection conduit, the stabilization chamber, the flow-stabilizing element, the fluidic oscillation chamber, the reflux element and the one or more pressure sensors or microphones are arranged.
the flow-stabilizing element is spaced apart from the peripheral wall of the stabilization chamber in such a way as to create passages for the gas around said flow-stabilizing element. The flow of gas thus crosses the stabilization chamber by going round the flow-stabilizing element, that is to say passing along both sides of the flow-stabilizing element.
a gas evacuation conduit is fluidically connected to the second gas outlet orifice of the oscillation chamber in such way as to recover the gas leaving the oscillation chamber.

The invention relates to a device for monitoring oxygen therapy, said device comprising a fluidic oscillation flowmeter according to the invention.

Moreover, the invention also relates to oxygen therapy equipment comprising:
a source of respiratory gas, for example a gas distribution appliance or a gas cylinder,
a gas distribution interface for distributing the respiratory gas to a patient, such as nasal cannulas or a breathing mask, and
a monitoring device with fluidic oscillation flowmeter according to the invention.

### BRIEF DESCRIPTION OF THE DRAWINGS

The invention will now be better understood from the following detailed description, given by way of non-limiting illustration, with reference to the attached figures among which:

FIG. 1 is a diagram showing the operating principle of a fluidic oscillation flowmeter according to the invention,

FIG. 2 is a three-dimensional representation of a fluidic oscillation flowmeter according to the invention, similar to that of FIG. 1,

FIG. 3 is a diagram of an embodiment of a device for monitoring oxygen therapy, comprising a fluidic oscillation flowmeter according to the invention,

FIG. 4 shows oxygen therapy equipment including a monitoring device as per FIG. 3 and a fluidic oscillation flowmeter according to the invention,

FIG. 5 shows the placement of the sensors and the geometry tested during the simulation tests,

FIG. 6 is a graph of the simulation tests carried out to show the importance of the precise positioning of the measurement orifices,

FIG. 7 is a graph of the simulation tests carried out to show the importance of the precise positioning of the measurement orifices,

FIG. 8 is a graph of the simulation tests carried out to show the importance of the precise positioning of the measurement orifices,

FIG. 9 is a graph of the simulation tests carried out to show the importance of the precise positioning of the measurement orifices,

FIG. 10 is a graph of the simulation tests carried out to show the importance of the precise positioning of the measurement orifices,

FIG. 11 is a graph of the simulation tests carried out to show the importance of the precise positioning of the measurement orifices,

FIG. 12 is a graph of the simulation tests carried out to show the importance of the precise positioning of the measurement orifices, and

FIG. 13 schematically depicts the connection conduit of rectangular cross section in a fluidic oscillation flowmeter according to the invention.

### DESCRIPTION OF PREFERRED EMBODIMENTS

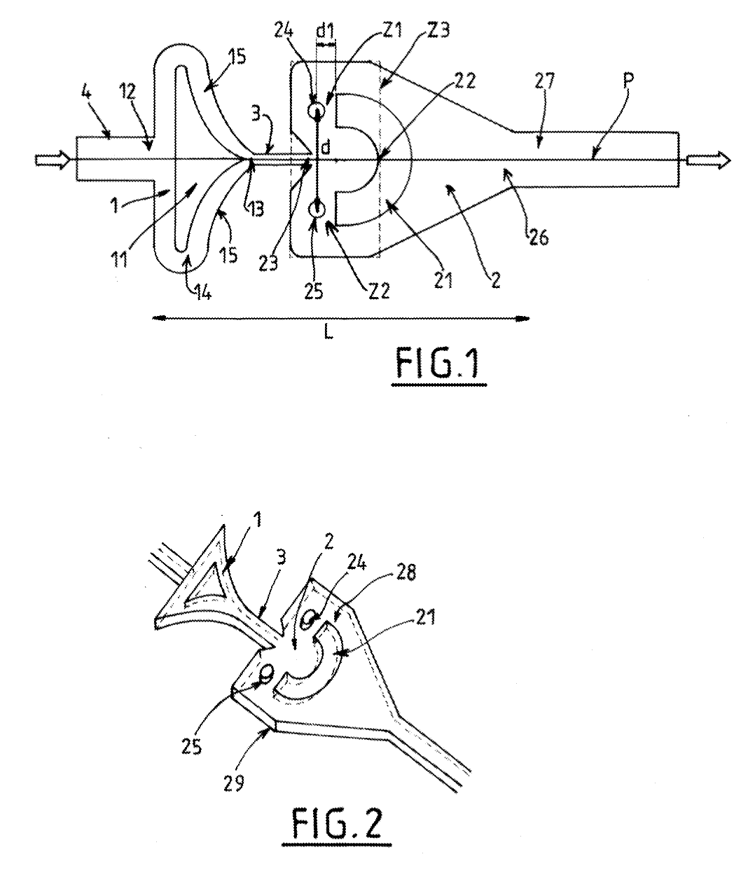

FIG. 1 is a diagram showing the operating principle of a fluidic oscillation flowmeter (seen from above) according to the invention. It comprises a stabilization chamber 1, in which a flow-stabilizing element 11, here of triangular cross section, is arranged, and an oscillation chamber 2 comprising a reflux element 21 having a semi-cylindrical shape, which is configured as an arc of a circle 22 in order to create an oscillating gaseous vortex. The vortex in fact oscillates between two zones Z1, Z2 situated schematically at the ends of the semi-cylinder forming the reflux element 21.

The reflux element 21 is sandwiched between two parallel walls 28, 29 delimiting the oscillation chamber 2 at the top and bottom respectively (FIG. 2), that is to say forming the ceiling and the floor of the oscillation chamber 2.

A connection conduit 3 fluidically connects the stabilization chamber 1 to the oscillation chamber 2, such that the gas entering the stabilization chamber 1 passes through the latter and then feeds the oscillation chamber 2. The connection conduit 3 opens into the latter in line with, that is to say facing or opposite, the reflux element 21 of semi-cylindrical shape, and this generates an oscillation of the flow and formation of vortices in the two aforementioned zones Z1 and Z2.

As will be seen, there is in fact a plane of symmetry P separating the whole system, in particular the connection conduit 3, the stabilization chamber 1, the flow-stabilizing element 11, the fluidic oscillation chamber 2 and the reflux element 21, into two equal and symmetrical parts with respect to this plane of symmetry P.

Such a configuration is known and described in the publication: Yves Le Guer; Jet confiné, dispersions fluide-particules et mélange chaotique; Engineering Sciences; Université de Pau et des Pays de l'Adour; 2005, and in the document WO 93/22627. According to said document, the depth of the channel feeding the oscillation chamber is 70 mm and its width is 10 mm.

However, such an architecture is not sufficient, especially if the positioning of the pressure taps of the flowmeter is not chosen with care. Indeed, if the pressure taps are poorly positioned, a flowmeter equipped with such a system will not be efficient enough.

In other words, it has been found that certain dimensions are particularly important and have to be respected in order to obtain an effective and precise flowmeter, in particular the positioning of the pressure taps and the dimensions (h₀, l₀) of the connection conduit 3 fluidically connecting the stabilization chamber 1 to the oscillation chamber 2.

Thus, during tests carried out in the context of the present invention, it was shown that, in order to ensure efficient measurement of the variation of the gas pressure, as a function of time, in the reflux chamber 2 in which the gaseous flux oscillates to form gaseous vortices in the zones Z1, Z2, the measurement site of the pressure sensors or microphones must be chosen with precision, namely the two measurement orifices 24, 25 to which the pressure sensors or microphones (not shown) are connected.

According to the invention, the two measurement orifices 24, 25 must be arranged, in the ceiling 28 (or in the floor 29) of the reflux chamber 2, that is to say approximately above the zones Z1, Z2 where the vortices form, and especially symmetrically with respect to the plane of symmetry P of the flowmeter, imperatively with a distance d between them (measured between the axes or centers of the measurement orifices) of between 0.5 and 15 mm (cf. FIG. 1), preferably between 0.5 and 10 mm, for example of the order of 1 to 6 mm.

The two measurement orifices 24, 25, preferably connected to microphones, are situated on an axis perpendicular to the plane of symmetry P, preferably in the zone Z3 shown by broken lines in FIG. 1, as is explained below.

Indeed, the principle of a fluidic oscillation flowmeter as described in the abovementioned publications does not permit an efficient flowmeter to be obtained unless the positioning of the two measurement orifices 24, 25 is chosen with care.

Indeed, the positioning of the two measurement orifices 24, 25 with respect to each other, and with respect to other elements of the geometry of the flowmeter system, plays an important role in the perception of the oscillation frequency of the pressure of the vortex and consequently influences the precision of the calculation of the flow rate based on the pressure values measured by these sensors.

It is thus also essential to specifically dimension the connection conduit 3 which conveys the gas flow into the reflux chamber 2 where the two measurement orifices 24, 25 are situated and connected preferably to microphones (not shown), as is explained below.

It must be emphasized that the two measurement orifices 24, 25 are preferably closed by a fluidically leaktight membrane so as to ensure the correct function of the microphones. Indeed, the pressure in the oscillation chamber 2 is transmitted to the sensors or to the microphones via the two orifices 24, 25 and through the membranes which cover these two orifices 24, 25. The membrane preferably has a very small thickness in the area of the sensors 24 and 25, typically of the order of about 50 to 500 μm; elsewhere, its thickness can be between 1 and 2 mm, or even more.

Indeed, the gas flow circulates in the direction of the arrows (=>) shown in FIG. 1. The flow of gas, for example oxygen or oxygen-enriched air, arrives via an inlet channel 4 fluidically connected to the first inlet orifice 12 of the stabilization chamber 1 and enters said stabilization chamber 1 via this first inlet orifice 12.

Within the stabilization chamber 1, the flow is subjected to stabilization by the flow-stabilizing element 11, which has a cross section approaching that of a triangle with its base oriented opposite the mouth of the inlet channel 4, hence facing the first inlet orifice 12. In fact, the cross section of the flow-stabilizing element 11 is slightly concave as it approaches the inlet 13 of the conduit 3.

The gas flow goes round the flow-stabilizing element 11 by flowing through the passages 15 formed on each side of the latter. The passages 15 are in fact delimited by the outer surface of the flow-stabilizing element 11 and by the inner peripheral wall 14 of the stabilization chamber 1. In other words, the flow-stabilizing element 11 is spaced apart from the peripheral wall 14 of the stabilization chamber 1 in such a way as to create passages 15 for the gas around said flow-stabilizing element 11.

The gas flow then leaves the stabilization chamber 1 via the first outlet orifice 13 and is conveyed through the connection conduit 3 which fluidically connects the first outlet orifice 13 of the stabilization chamber 1 to the second inlet orifice 23 of the oscillation chamber 2.

The first and second inlet orifices 12, 13 and the first and second outlet orifices 13, 26 are arranged symmetrically with respect to the plane of symmetry P, as can be seen in FIG. 1.

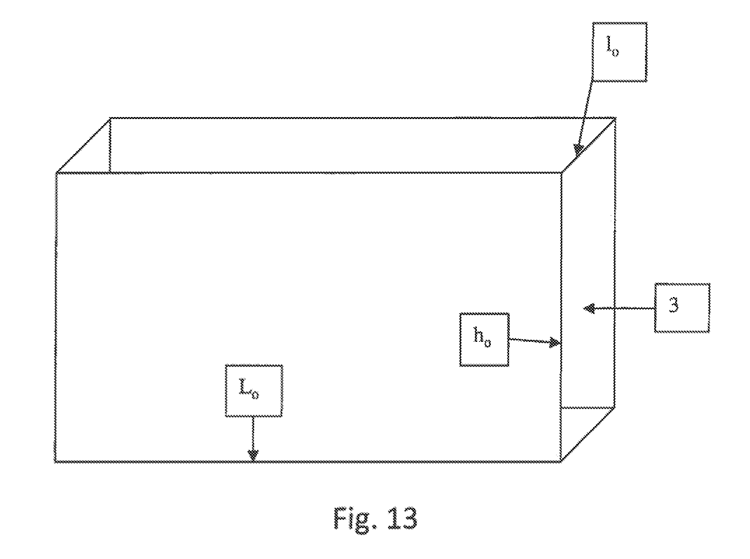

According to the present invention, in order to be able to ensure effective measurements, the connection conduit 3 also has to be configured and dimensioned in a specific way. Thus, according to the invention, the connection conduit 3 is of rectangular cross section, that is to say it has the general shape of a parallelepiped with a width l₀ and a height h₀ such that: 6.5·l₀≧h₀≧3·l₀, where the width l₀ is for example 0.5 to 1.5 mm, more preferably between 0.8 and 1.3 mm; this is illustrated in FIG. 13. Advantageously, h₀ and l₀ are chosen such that: h₀≧3.1·l₀, preferably: h₀≧3.5·l₀ and/or 6·l₀≧h₀.

In other words, by choosing a connection conduit 3 whose width is small in relation to its height, it will be possible to obtain a two-dimensional laminar flow with a sufficiently high speed, which will favor its oscillation in the reflux chamber 2.

Moreover, it is also preferable to observe a length L₀ of the connection conduit 3 in relation to its width l₀, such that 2·l₀≦L₀≦10·l₀, preferably with: 3·l₀≦L₀≦7·l₀.

Generally, as illustrated in FIG. 1, the flow then enters the oscillation chamber 2 and there impacts the reflux element 21 of semi-cylindrical shape, and this creates the oscillating vortex between the two zones Z1 and Z2, as has been explained above.

The gas then continues its travel through the oscillation chamber 2 before leaving the latter through a gas evacuation conduit 27, which is fluidically connected to the second gas outlet orifice 26 of the oscillation chamber 2.

It will thus be understood that, starting from a speed symmetrical in two dimensions, a vortex is created whose location (zones Z1 and Z2) will oscillate with a frequency proportional to the value of the flow rate of the fluid that circulates there. By placing microphones or pressure measurement members/sensors outside the path of the fluid, that is to say above zones Z1, Z2 where the vortices form, it is possible to measure the presence or absence of a drop in pressure of the gas.

With the flowmeter of the invention, the flow rate of the circulating gas can be measured in a non-intrusive, miniaturized and inexpensive manner, with a pressure drop that is limited by comparison with a flowmeter having a throttle.

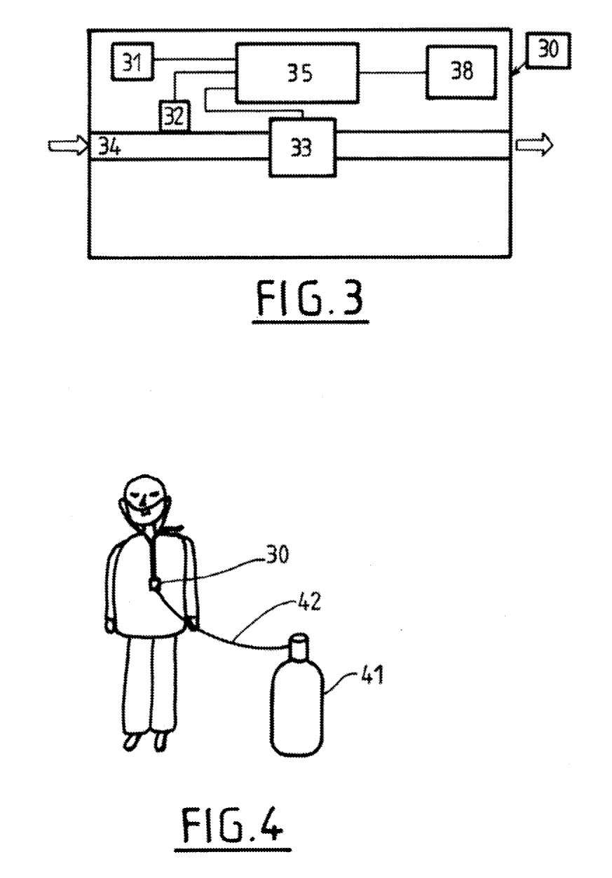

The whole system is accommodated in a casing shown in FIG. 3, in particular the connection conduit 3, the stabilization chamber 1, the flow-stabilizing element 11, the fluidic oscillation chamber 2, the reflux element 21 and the one or more pressure sensors or microphones.

Moreover, control means 35 such as an electronic card with microprocessor, for example a microcontroller, are connected electrically to the pressure sensors or microphones in such a way as to collect and exploit the pressure measurements by extracting their oscillation frequency and then deducing therefrom a gas flow rate, as is illustrated in FIG. 3 and explained below.

FIG. 2 is a three-dimensional representation of the flowmeter from FIG. 1, showing the location of the measurement orifices 24, 25 in the ceiling 28 of the reflux chamber 2.

FIG. 3 is a diagram of an embodiment of a device for monitoring oxygen therapy, comprising a fluidic oscillation flowmeter 33 according to the invention, comprising a casing 30 incorporating a first absolute pressure sensor 31 for measuring the ambient pressure, that is to say the atmospheric pressure, and a second absolute pressure sensor 32 for measuring the absolute pressure in the cannula 34, which sensor 32 is placed in direct contact with the cannula 34, before or after the fluidic oscillation flowmeter 33 according to the invention. The gas flow circulates in the cannula or conduit 34 in the direction of the arrows (=>).

A control and processor module 35, such as an electronic card, is connected electrically to the sensors 31, 32 and to the flowmeter 33 in such a way as to recover and process the measurements carried out by the sensors 31, 32 and the flowmeter 33. An energy source, such as an electric battery or a cell, is able to supply electric current to the control and processor module 35.

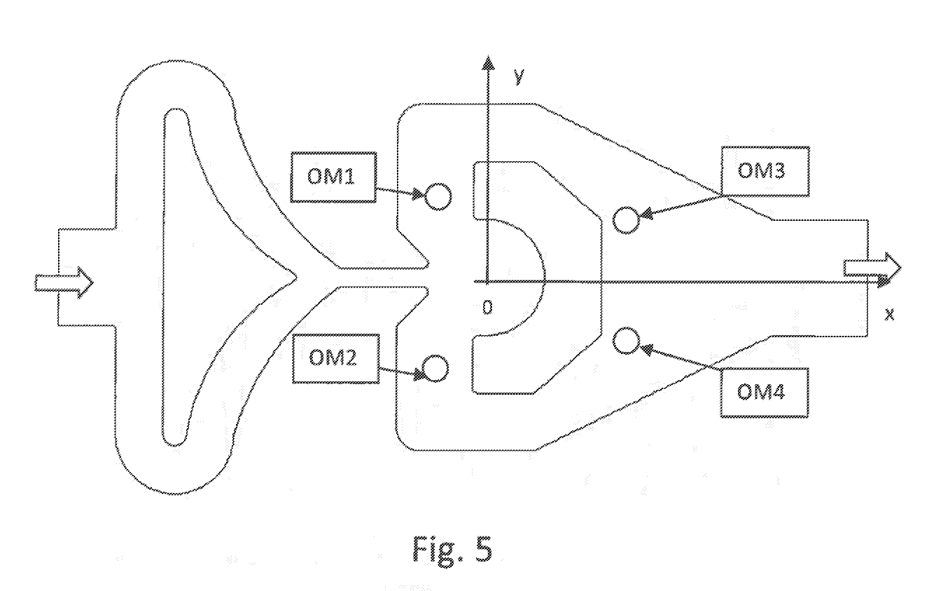

FIG. 4 shows a schematic view of oxygen therapy equipment according to the invention, comprising a source 41 of respiratory gas, here a gas cylinder, and a gas distribution interface 42 for distributing the respiratory gas to a patient, in this case in the form of nasal cannulas for example, and a monitoring device 30 with a fluidic oscillation flowmeter according to the invention, as shown schematically in FIG. 3.

In order to show the importance of the correct positioning of the measurement orifices 24, 25 according to the present invention, simulation tests have been carried out as explained below.

The geometry of the chosen flowmeter (cf. FIG. 1) for the 2D simulation tests and the simulation parameters are as follows:
modeling with Ansys Fluent software, and 2D mesh configuration with Ansys ICEM CFD on a base of 47,881 meshes.
condition at inlet limits: fixed inlet flowrate.
condition at outlet limits: débit sortant.
simulation in variable operating regime.
laminar model.

Several virtual measurement orifices OM1 to OM4 were positioned in order to evaluate the impact of their placement/positioning in terms of amplitude and frequency of the associated pressure signal. The positioning of the measurement orifices 24, 25 (designated OM1 to OM4 in FIG. 5), connected to the microphones or pressure measurement members measuring the presence or absence of a drop in pressure, was varied.

TABLE
Different positions of the axes of the measurement orifices
with respect to the origin of the axes situated at the
centre of the inner circle of the cavity

| Position of the sensors | x (mm) | y (mm) |
|---|---|---|
| 1 | −1.5 | 2.25 |
| 2 | −1.5 | −2.25 |
| 3 | 3.5 | 2 |
| 4 | 3.5 | −2 |

After several tests of the positioning of the orifices OM1 to OM4 (cf. table above), it was noted that the pressure measurement allowing the gas flow rate to be deduced is not correct when the position of the acoustic measurements is not chosen with care.

Indeed, the tests demonstrated that the measurement orifices 24, 25, connected to the sensors, should be positioned symmetrically with respect to the plane P and at a distance d between the axes of the measurement taps of between 0.5 and 15 mm in order to guarantee quality measurements. The best results are obtained with a distance d between the axes of the measurement taps of between 1 and 10 mm, preferably between 1 and 6 mm, typically of the order of 3 to 5 mm.

Advantageously, their position is chosen preferably in the zone Z3 of FIG. 1, which are delimited in particular by the straight line perpendicular, at the inlet of the oscillation chamber, to the plane of symmetry P, and the straight line corresponding to the intersection between the plane of symmetry and the semi-cylindrical cavity of the reflux member 21.

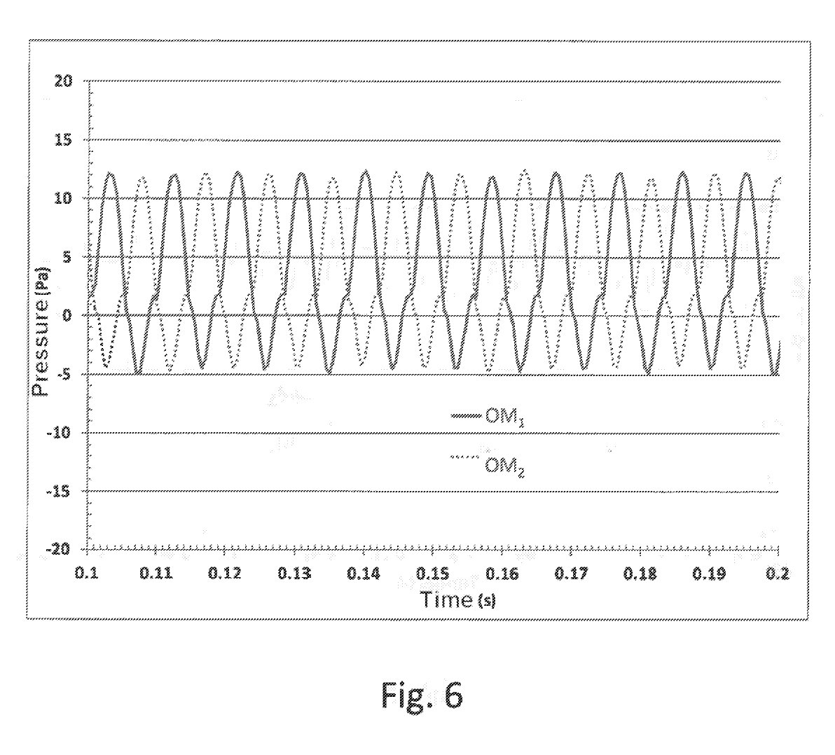

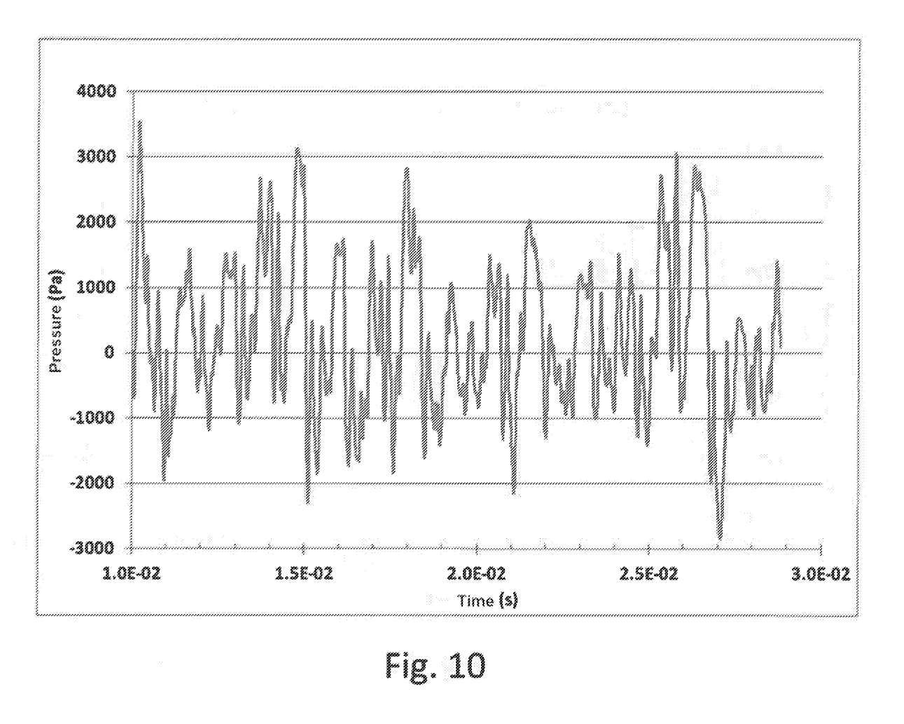

These results are shown in FIGS. 6 to 12. The oxygen flow rate of 5 l/min for FIGS. 6 to 9 and of 4 l/min for FIGS. 10 to 12.

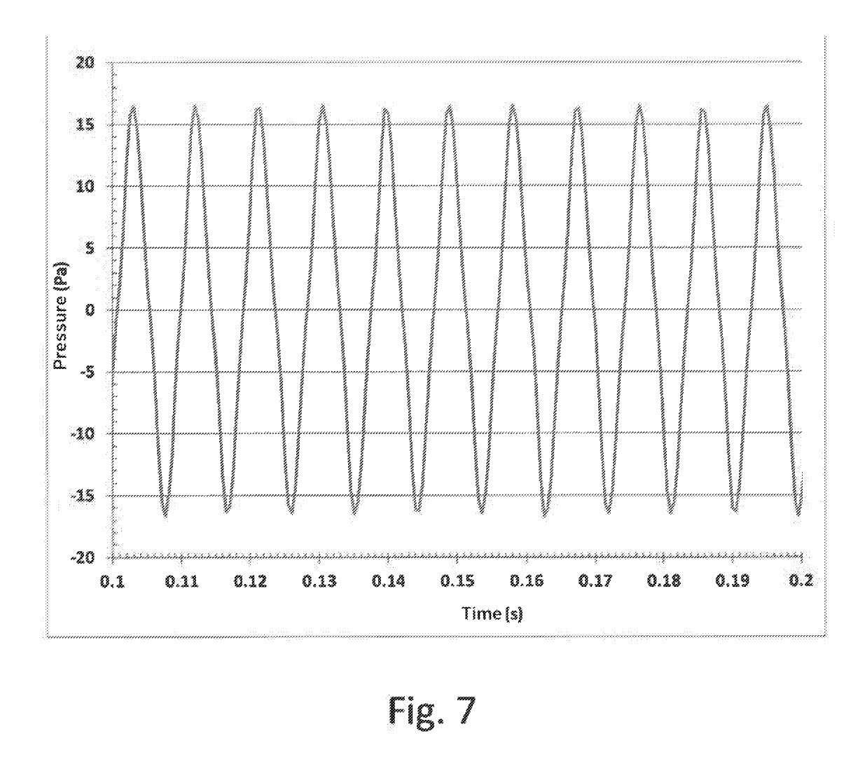

FIG. 6 shows the pressure signals (in Pa) as a function of time for the two measurement orifices OM1 and OM2, while FIG. 7 shows the pressure difference signal (in Pa) between the two measurement orifices OM1 and OM2 as a function of time (in seconds).

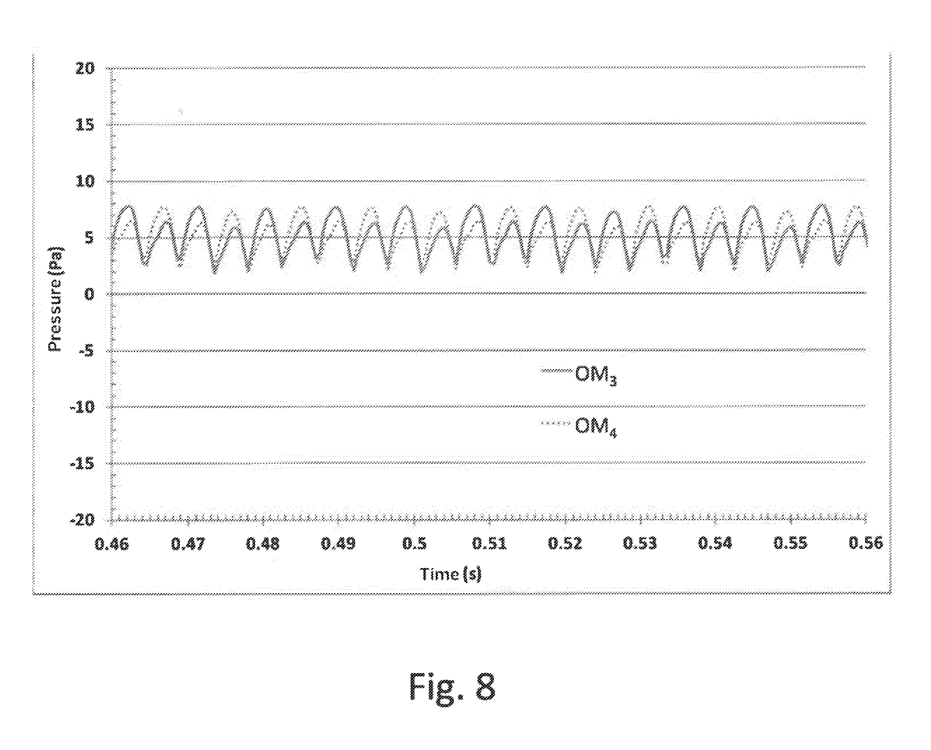

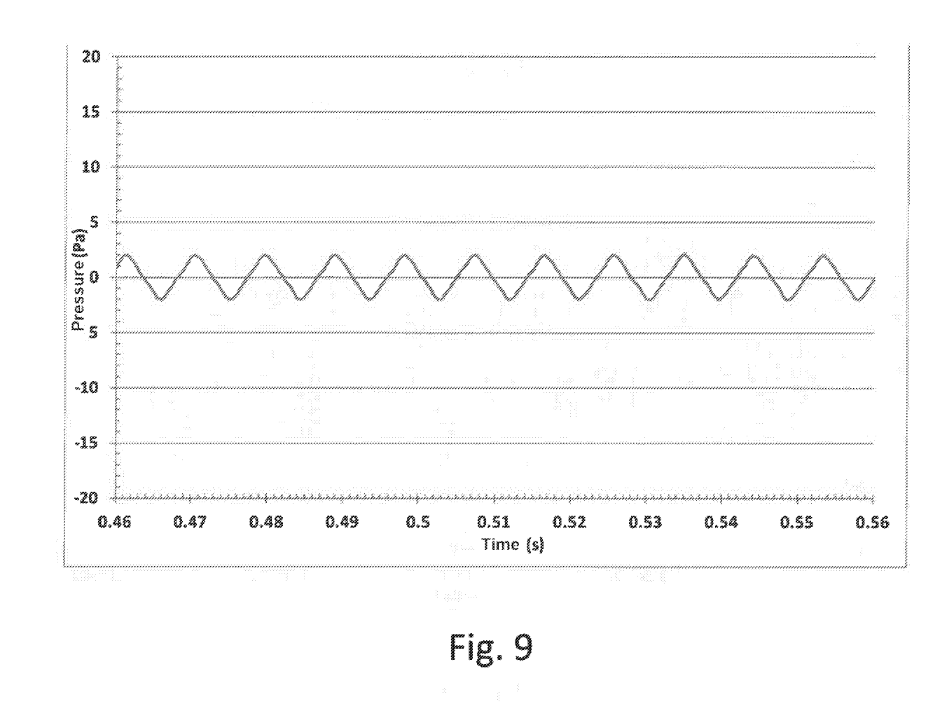

Similarly, FIG. 8 shows the pressure signals (in Pa) as a function of time (in seconds) for the two measurement orifices OM3 and OM4, while FIG. 9 shows the pressure difference signal (in Pa) between the two measurement orifices OM3 and OM4 as a function of time (sec).

In FIG. 6, the pressure signal is shown as a function of time (sec) seen by the sensors (microphones) connected to the two measurement orifices OM1 and OM2 placed in the zone Z3 and symmetrically with respect to the plane P. The exact placement of the two measurement orifices is defined in the table above. The amplitude of variation of the pressure signal (difference between the maximum and minimum values) as a function of time for the two measurement orifices OM1 and OM2 is 17 Pa. It is preferable to calculate the difference of the signals of the two sensors (FIG. 7). Indeed, this makes it possible to almost double the amplitude of variation of pressure (from 17 Pa to 34 Pa), but also to limit the noise which may appear on the pressure signals (noise due to the pressure variation associated with the respiratory frequency, for example).

In FIG. 8, the pressure signal is shown as a function of time seen by the two measurement orifices OM3 and OM4 placed outside the zone Z3. It will be noted that they are at the same phase of variation, whereas in the case of the two measurement orifices OM1 and OM2 they were in phase opposition. The amplitude of pressure variation for the two measurement orifices is only 5 Pa, i.e. much lower than the value of 17 Pa for the two measurement orifices OM1 and OM2. The difference of the two pressure signals for these two measurement orifices (see FIG. 9) remains almost at the same amplitude of 5 Pa, whereas the amplitude of the pressure difference for the two measurement orifices OM1 and OM2 is much higher at 34 Pa.

In conclusion, the two measurement orifices connected to the pressure sensors or microphones have to be placed in the zone Z3, but in particular symmetrically with respect to the plane P and with a distance d between them of between 0.5 and 10 mm, preferably of the order of 1.5 to 6 mm.

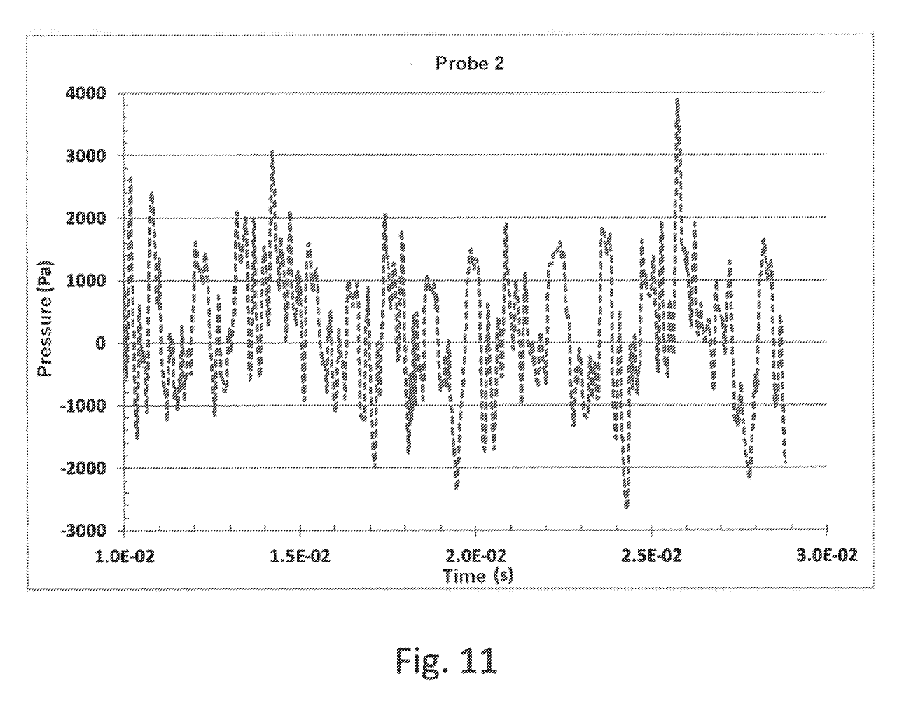

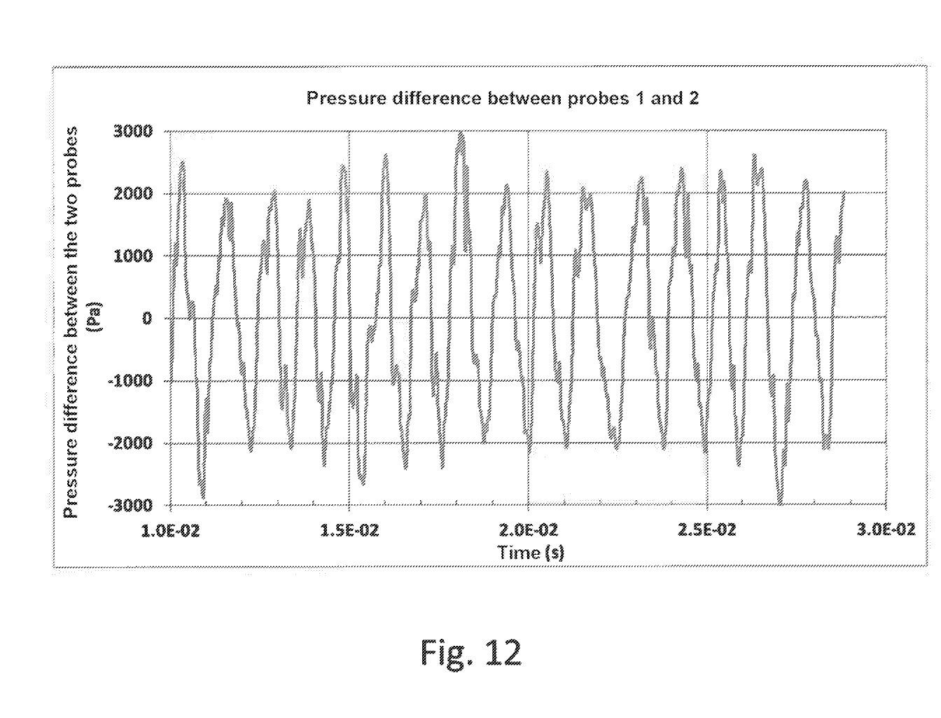

To show the importance of calculating the difference of the signals of the two pressure sensors or microphones in order to extract the oscillation frequency, FIGS. 10 and 11 show the pressure signals (in Pa) sensed separately by the measurement orifices OM1 (also called “Probe 1”) and OM2 (also called “Probe 2”) for an oxygen flowrate of 4 l/min, and also the difference of these two signals (FIG. 12).

If we examine separately the pressure signal picked up by the probes 1 and 2 (i.e. the measurement orifices OM1 and OM2), it is more difficult to extract the oscillation frequency, whereas the latter is clearly more visible from the difference of the signals. Hence the need to calculate the difference of the pressure signals and especially at a high flowrate, i.e. above 1 l/min. Moreover, this makes it possible to eliminate, where they exist, electronic noise on the two sensors and the ambient acoustic noise, and also the pressure variations induced by the respiratory frequency of the patient. All of these noises (electronic, acoustic, patient's breathing) in fact disturb the pressure signals of the two sensors in the same way. It is therefore preferable to eliminate them by effecting a difference between the pressure signals.

The fluidic oscillation flowmeter according to the invention is particularly well adapted for use in a device for monitoring oxygen therapy of a patient at home, said monitoring device being connected, on the one hand, to a source of respiratory gas and, on the other hand, to a gas distribution interface, such as a breathing mask, a nasal cannula or similar, serving to supply respiratory gas, typically gaseous oxygen, to the patient.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A fluidic oscillation flowmeter comprising:
a stabilization chamber (1) comprising a flow-stabilizing element (11),
an oscillation chamber (2) comprising a reflux element (21) configured for and adapted to create at least one oscillating gaseous vortex in the oscillation chamber (2), said reflux element (21) being arranged between two parallel walls (28, 29) delimiting the oscillation chamber (2),
a connection conduit (3) fluidically connecting the stabilization chamber (1) to the oscillation chamber (2), and
a plane of symmetry (P) separating the connection conduit (3), the stabilization chamber (1), the flow-stabilizing element (11), the fluidic oscillation chamber (2) and the reflux element (21) into two equal and symmetrical parts with respect to said plane of symmetry (P),
wherein:
one of said two parallel walls (28, 29) delimiting the oscillation chamber (2) comprises two measurement orifices (24, 25) which are arranged symmetrically with respect to the plane of symmetry (P) and on an axis perpendicular to the plane of symmetry (P) and are separated from each other by a distance (d) of between 0.5 mm and 15 mm, and
the connection conduit (3) has a rectangular cross section of width l₀ and height h₀ such that 6.5·l₀≧h₀≧3·l₀.

2. The flowmeter of claim 1, wherein the width l₀ and the height h₀ of the connection conduit (3) are such that:
h₀≧3.1·l₀, preferably h₀≧3.5·l₀, and/or
6·l₀≧h₀.

3. The flowmeter of claim 2, wherein the connection conduit (3) of rectangular cross section has a length L₀ such that: 2·l₀≦L₀≦10·l₀.

4. The flowmeter of claim 1, wherein the two measurement orifices (24, 25) are separated by a distance (d) of between 0.5 mm and 10 mm.

5. The flowmeter of claim 1, wherein the flow-stabilizing element (11) has a cross section in the general shape of a triangle and/or the reflux element (21) comprises a part (22) of semi-cylindrical cross section arranged facing the connection conduit (3).

6. The flowmeter of claim 1, wherein:
the stabilization chamber (1) comprises a first inlet orifice (12) and a first outlet orifice (13), which are arranged on the plane of symmetry (P) and
the oscillation chamber (2) comprises a second inlet orifice (23) and a second outlet orifice (26), which are arranged on the plane of symmetry (P), and
the connection conduit (3) fluidically connects the first outlet orifice (13) of the stabilization chamber (1) to the second inlet orifice (23) of the oscillation chamber (2).

7. The flowmeter of claim 1, further comprising one or more pressure sensors or microphones attached to said two measurement orifices (24, 25) in such a way as to be able to measure the pressure in the oscillation chamber (2).

8. The flowmeter of claim 1, wherein the two measurement orifices (24, 25) are closed by a fluidically leaktight membrane.

9. The flowmeter of claim 6, wherein an inlet channel (4) is fluidically connected to the first inlet orifice (12) of the stabilization chamber (1).

10. The flowmeter of claim 7, further comprising a casing within which the connection conduit (3), the stabilization chamber (1), the flow-stabilizing element (11), the fluidic oscillation chamber (2), the reflux element (21) and the one or more pressure sensors or microphones are arranged.

11. The flowmeter of claim 1, wherein the flow-stabilizing element (11) is spaced apart from a peripheral wall (14) of the stabilization chamber (1) in such a way as to create a passage (15) for the gas around said flow-stabilizing element (11).

12. A device for monitoring oxygen therapy, comprising the fluidic oscillation flowmeter of claim 1.

13. Oxygen therapy equipment comprising:
a source of respiratory gas,
a gas distribution interface for distributing the respiratory gas to a patient, and
a monitoring device with the fluidic oscillation flowmeter of claim 12.
